(19) United States
(12) Patent Application Publication
(10) Pub. No.: US 2024/0182206 A1
(43) Pub. Date: Jun. 6, 2024

(54) RETAINING TOOL, USE METHOD OF RETAINING TOOL, SYRINGE TRANSPORT TOOL SET, AND TRANSPORTATION METHOD OF SYRINGE

(71) Applicant: THREEBOND CO., LTD., Hachioji-shi, Tokyo (JP)

(72) Inventors: Misako HIROTA, Hachioji-shi, Tokyo (JP); Makoto KATO, Hachioji-shi, Tokyo (JP); Takashi NEMOTO, Hachioji-shi, Tokyo (JP)

(21) Appl. No.: 18/552,528

(22) PCT Filed: Mar. 25, 2022
(86) PCT No.: PCT/JP2022/014298

§ 371 Date: Sep. 26, 2023

(30) Foreign Application Priority Data
Apr. 15, 2021 (JP) 2021-069244

(51) Int. Cl. B65D 25/10; A61M 5/00; B65D 81/107; B65D 81/18; B65D 81/38

(52) U.S. Cl. CPC B65D 25/108; A61M 5/002; B65D 81/107; B65D 81/18; B65D 81/3825

(57) ABSTRACT

A retaining tool is to be arranged in a housing space of a packing container and has: an insertion portion into which a syringe containing a material storage container that stores a viscous material can be inserted in an upright posture; and a solid portion that is provided around the insertion portion and forms the insertion portion. The solid portion is formed from a first end portion at which the insertion portion is positioned in the direction in which the viscous material is inserted into the insertion portion to a second end portion that is on the opposite side from the first end portion; includes a non-crosslinked polyethylene foam; and prevents bubbling when the viscous material as frozen at 0° C. or below thaws. The present invention also provides a usage method for the retaining tool, a syringe transport tool set, and a transport method for a syringe.

### TECHNICAL FIELD

The present invention relates to a retaining tool, a use method of a retaining tool, a syringe transport tool set, and a transportation method of a syringe.

### BACKGROUND ART

There are various types of viscous materials, and examples thereof include a one-liquid type curable resin and a resin of a two-liquid curable type, and the like. Among them, some one-liquid curable resins undergo progression of curing even at room temperature, and there are some one-liquid curable resins need to be maintained at the state of 0° C. or lower in the stages from production to transportation and safekeeping, and the like.

As a technique for maintaining the temperature, for example, during transportation of a one-liquid curable resin at 0° C. or lower, there is a technique for disposing dry ice inside a container using a heat insulating material so as to maintain the temperature of the curable silicone composition at −40 to 0° C. (see Patent Literature 1).

### CITATION LIST

### Patent Literature

Patent Literature 1: JP 4503457 B2

### SUMMARY OF INVENTION

The viscous material held at 0° C. or lower needs to be thawed at the time of use. The present inventors are concerned about a matter that air bubbles are generated in the viscous material depending on a thawing method when the viscous material cooled to 0° C. or less is thawed to about room temperature in a state of being housed in a container.

Therefore, an object of the present invention is to provide a retaining tool, a use method of the retaining tool, a syringe transport tool set, and a transportation method of the syringe that prevent or suppress generation of air bubbles when a viscous material frozen to 0° C. or less is thawed.

A retaining tool according to one aspect of the present invention for solving the above problems includes an insertion portion and a solid portion. The insertion portion is installed in the housing space of the packing container, and is configured to allow insertion of the syringe in a state where the syringe including a material storage container that houses the viscous material is erected. The solid portion is provided around the insertion portion and forms the insertion portion. The solid portion is formed from a first end portion on the side where the insertion portion is located to a second end portion on the opposite side of the first end portion in the direction in which the viscous material is inserted into the insertion portion, and contains non-crosslinked foamed polyethylene.

Furthermore, a use method of a retaining tool according to one aspect of the present invention includes thawing the syringe from −40° ° C. to room temperature in a state where the syringe is inserted into the insertion portion.

Furthermore, one aspect of the present invention is a syringe transport tool set including a surrounding member that surrounds the syringe and a cold storage agent. Furthermore, one aspect of the present invention is a transportation method of the syringe.

### BRIEF DESCRIPTION OF DRAWINGS

FIG. 1 is a perspective view schematically illustrating a syringe transport tool set according to one embodiment of the present invention.

FIG. 2 is a plan view illustrating an inside of a packing container in the syringe transport tool set of FIG. 1.

FIG. 3 is a perspective view of a cold storage agent constituting the syringe transport tool set.

FIG. 4 is a front view illustrating the cold storage agent.

FIG. 5 is a perspective view illustrating a retaining tool and a syringe to be housed in the syringe transport tool set.

FIG. 6 is a front view of FIG. 5.

FIG. 7 is a graph illustrating temperature changes when syringes housing a viscous material are set in retaining tools according to an example and a comparative example and thawed in Experiment 1.

FIG. 8 is a graph illustrating a temperature change of a viscous material with respect to a temporal change of an outside air temperature in Experiment 2.

### DESCRIPTION OF EMBODIMENT

Hereinafter, modes for carrying out the present invention will be described in detail with reference to the drawings. The embodiment shown herein is the drawings. exemplified to embody the technical idea of the present invention, and do not limit the present invention. Furthermore, other modes that can be implemented, examples, operation techniques, and the like that can be conceived by those skilled in the art or the like without departing from the gist of the present invention are all included in the scope and gist of the present invention and included in the invention described in the claims and the scope of equivalents thereof.

In addition, for convenience of illustration and ease of understanding, the drawings attached to the present specification may be schematically represented by changing a scale, a dimension ratio of length and width, a shape, and the like from actual ones as appropriate, but are merely examples, and do not limit the interpretation of the present invention.

Furthermore, In the following description, ordinal numerals such as “first” and “second” will be given to provide description, but are used for convenience and do not define any order unless otherwise specified.

Furthermore, in the following, a coordinate system is used in the drawings and the description thereof. X and Y are planar directions in which a surrounding member 200, a cold storage agent 300, and a retaining tool 400 are placed, and are referred to as a first direction X, a second direction Y, or a planar direction XY. Z corresponds to a height direction of the packing container 100 and the retaining tool 400, and is referred to as a height direction Z.

### (Syringe)

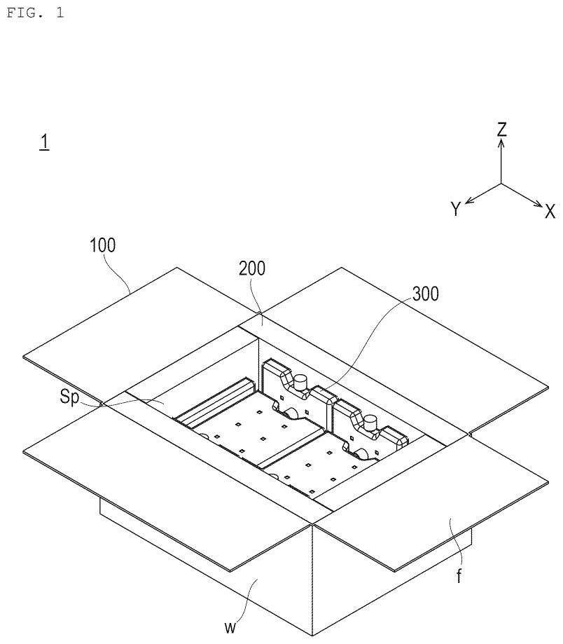

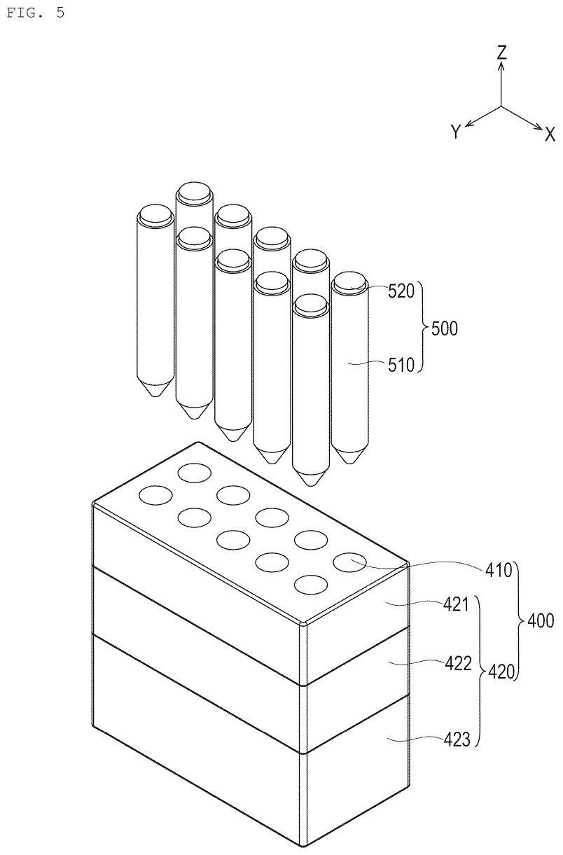

A syringe 500 is used by setting a viscous material in an application device such as a dispenser or the like. The syringe 500 includes, as illustrated in FIG. 5, a material storage container 510 and a lid portion 520. The syringe 500 can be installed in a housing space Sp (see FIG. 1) of the packing container 100.

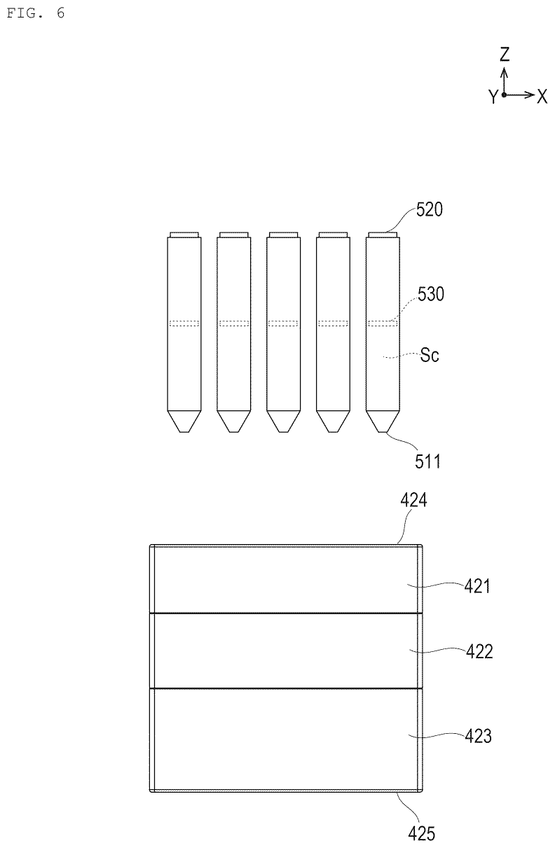

The material storage container 510 is configured to provide a semi-closed space Sc that houses the viscous material. The material storage container 510 includes, as illustrated in FIG. 6, an opening portion 511 through which the viscous material can be discharged from the semi-closed space Sc. The material storage container 510 can be provided with a plunger 530 at an intermediate portion in the height direction Z as illustrated in FIG. 6.

The plunger 530 can move the viscous material filled in the semi-closed space Sc in a sealed state toward the opening portion 511 by being movable in the height direction Z. The plunger 530 is provided, in the present embodiment, at the center in the height direction Z of the material storage container 510, but the position of the plunger is not limited to the center in the height direction Z as long as the viscous material can be stored in the semi-closed space Sc.

The material storage container 510 is configured such that a pointed shape or a curved surface shape is provided at the tip of the substantially cylindrical shape in the height direction Z. However, the specific shape is not limited to the above as long as a semi-closed space that houses the viscous material can be formed.

The lid portion 520 is attached near an end portion opposite to the opening portion 511 in the height direction Z in a state where the viscous material is housed in the semi-closed space Sc of the material storage container 510. The syringe 500 can install the material storage container 510 in the dispenser or the application device in a state where the viscous material is stored in the semi-closed space Sc of the material storage container 510 and the lid portion 520 is removed from the material storage container 510.

Examples of the viscous material transported by a syringe transport tool set 1 according to the present embodiment include, specifically, an adhesive, a sealant, a coating agent, a conductive adhesive, a thermally conductive resin, a flame-retardant resin, and the like, and among them, an adhesive, a sealant, a conductive adhesive, and conductive a thermally resin are preferable, and an adhesive and a conductive adhesive are exemplified as being particularly preferable, since the temperature fluctuation of the viscous material with respect to the temperature fluctuation of the outside air can be further suppressed.

The component of the viscous material is not particularly limited, and examples thereof include a urethane resin, an epoxy resin, an oxetane resin, a (meth) acrylic resin, a silicone resin, and the like, and among them, an epoxy resin, an oxetane resin, and a (meth) acrylic resin are preferable, and an epoxy resin is particularly preferable. By using more preferable components, it is possible to further suppress the temperature fluctuation of the viscous material with respect to the temperature fluctuation of the outside air.

The viscosity of the viscous material is not particularly limited, but is preferably in the range of 0.1 to 150 Pa·s, more preferably 1 to 75 Pa·s, and particularly preferably 5 to 30 Pa·s. Within the above range, the temperature fluctuation of the viscous material with respect to the temperature fluctuation of the outside air can be further suppressed. The measurement of the viscosity in the present invention is not particularly limited, but for example, 2.0 mL of the viscous material is weighed after stirring with a polytetrafluoroethylene rod, and the viscosity is measured using Brookfield (model number: DV-2+Pro) in a state where the temperature is set to 25° C. by a temperature control device. As measurement conditions, CPE-41 (3°× R2.4) was used for a cone rotor, the rotation speed was set to 10 rpm, and the viscosity after 3 minutes was set to “viscosity (Pa·s)”.

### (Syringe Transport Set)

The syringe transport tool set 1 includes, as illustrated in FIG. 1, the packing container 100, a surrounding member 200, and the cold storage agent 300. The syringe 500 transported by the syringe transport tool set 1 can be installed on the retaining tool 400 in a state where the lid portion 520 is attached to the material storage container 510. Hereinafter, description will be made.

### (Packing Container)

As the packing container 100 illustrated in FIG. 1, a known cardboard or the like can be used. The packing container 100 can be configured by joining materials such as waste paper, pulp, or the like with glue or the like. Furthermore, the packing container 100 can form a closed space that stores the viscous material by maintaining flaps f provided continuously in both the upper and lower sides in the height direction Z of a side surface w in a state of being closed with an adhesive tape or the like in each of the upper and lower sides.

### (Surrounding Member)

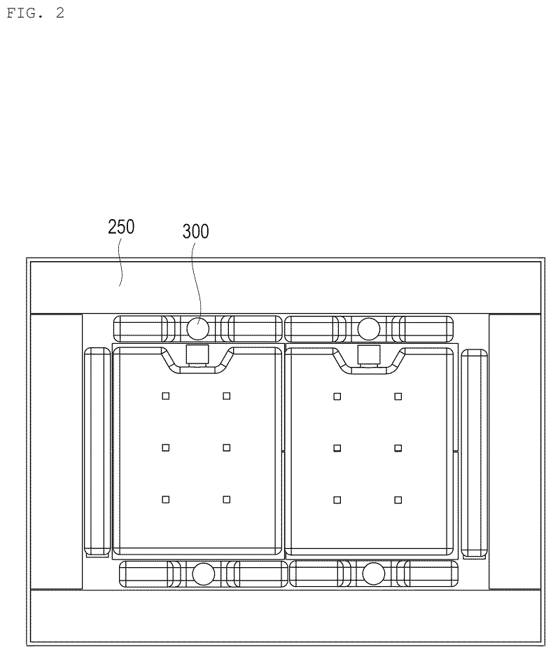

The surrounding member 200 is, as illustrated in FIG. 1, FIG. 2, installed in the housing space Sp of the packing container 100 and surrounds the syringe 500. The surrounding member 200 includes the buffer member that mitigates an external force applied to the syringe 500 when the syringe 500 is transported. The surrounding member 200 is configured to have a thermal conductivity of 0.022 W/m·K or less (JIS A9521). Note that the thermal conductivity is a value according to JIS A9521. As the surrounding member 200, a building heat insulating material containing polystyrene or the like containing air bubbles can be used. The surrounding member 200 is configured in a substantially rectangular parallelepiped shape in the present embodiment. However, the specific shape is not limited to a rectangular parallelepiped as long as the temperature of the viscous material can be easily maintained. The internal volume of the surrounding member 200 is, for example, 318 mm×415 mm×330 mm.

By being configured as described above, the surrounding member 200 can be made lighter than the vacuum heat insulating material, and can contribute to increasing the viscous material that can be transported at one time. The weight of the surrounding member 200 can be set to about 4 kg while the vacuum heat insulating material having the same volume is about 13 kg. The weight of the surrounding member 200 per one can be configured to be about 760 g as an example. The vacuum heat insulating material described above can be configured such that, for example, a glass wool core material is wrapped with a gas barrier film and sealed in a vacuum state.

### (Cold Storage Agent)

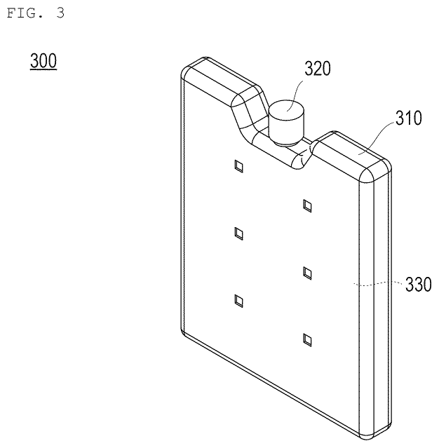

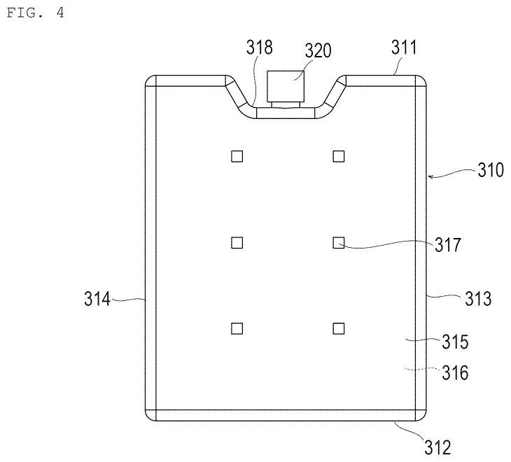

The cold storage agent 300 cools the syringe 500 housed in the packing container 100. The cold storage agent 300 includes, as illustrated in FIG. 3 and FIG. 4, a content storage container 310, a cap 320, and a content 330. The weight of the cold storage agent 300 per one cold storage agent can be configured to be, for example, about 1300 g.

The content storage container 310 is configured to be installable between the syringe 500 and the surrounding member 200 in the housing space Sp of the packing container 100. The content storage container 310 is provided with a housing space capable of housing the content 330.

The content storage container 310 is configured in a shape such as a rectangular parallelepiped as an example so as to be housed in the packing container 100 in various postures. That is, surfaces 311 to 316 of the content storage container 310 are configured as flat surfaces. However, the shape of the content storage container 310 is an example, and the specific shape is not limited to a rectangular parallelepiped as long as being able to be housed in various postures.

The content storage container 310 is configured such that recessed portions 317 are provided on surfaces 311, 316 having a relatively large area among six surfaces 311 to 316 illustrated in FIG. 4. In addition, the contents storage container 310 is configured such that a recessed portion 318 is provided in a part of the hexahedron such that the cap 320 to lid hardly protrudes from the content storage container 310 in a state where the content 330 which is a refrigerant is housed in the housing space. However, as long as the viscous material to be transported can be maintained at a predetermined temperature, the recessed portions 317, 318 may not be provided in the material storage container.

The material constituting the content storage container 310 is not particularly limited as long as the material does not deform so much that the volume significantly changes during transportation or is difficult to deform, but examples thereof include polyethylene, polypropylene, polyethylene terephthalate, and the like. The content storage container 310 may adopt either a bag type that is relatively easily deformed by application of an external force or a hard type that is relatively hardly deformed and easily maintains a predetermined shape even when an external force is applied, but it is preferable to adopt the hard type from the viewpoint of difficulty in deformation as described above.

The content 330 cools the syringe 500 housed in the packing container 100. The content 330 is configured to have a melting point of −30° C. or lower. The lower limit value of the melting point of the content 330 is not particularly limited, but is preferably −50° C. The content 330 can be configured to include an aqueous solution such as an inorganic salt.

### (Retaining Tool)

The retaining tool 400 is used to retain the syringe 500. The retaining tool 400 includes, as illustrated in FIG. 5, an insertion portion 410 and a solid portion 420.

The insertion portion 410 is provided in the solid portion 420 and is configured to allow insertion of the syringe 500 in a state where the syringe 500 is erected. In the insertion portion 410, a cross section orthogonal to the longitudinal direction (height direction Z) in accordance with the shape of the syringe 500 is configured in a perfectly circular shape. However, the shape of the insertion portion is not limited to this as long as the syringe 500 can be retained, and the insertion portion may be configured by another circle other than a perfect circle such as an ellipse or the like or a polygon.

The solid portion 420 is provided around the insertion portion 410 and is configured to form the insertion portion 410. The solid portion 420 is, as illustrated in FIG. 6, configured to be formed from a first end portion 424 where the insertion portion 410 is located to a second end portion 425 on the opposite side of the first end portion 424 in the direction in which the viscous material is inserted.

The solid portion 420 is configured to contain non-crosslinked foamed polyethylene. The linear expansion coefficient of the non-crosslinked foamed polyethylene constituting the solid portion 420 is 0.1 to 10×10⁻⁴ cm/cm·° C., and preferably 1 to 7×10⁻⁴ cm/cm·° C.

The solid portion 420 is, as illustrated in FIG. 5 in the present embodiment, configured in a layer shape so as to sequentially provide a first layer 421, a second layer 422, and a third layer 423 in the insertion direction (height direction Z) in which the syringe 500 is inserted. However, as long as the insertion portion can be formed, the number of layers is not limited thereto, and may be a single layer.

The solid portion 420 is configured such that the second end portion 425 becomes a flat surface so that the retaining tool 400 can be easily installed as illustrated in FIG. 6.

### (Transportation Method of Syringe)

Next, a transportation method of the syringe 500 using the syringe transport tool set according to the present embodiment will be described.

First, a packing container 100 such as cardboard or the like is prepared, and one of the flap portions (the lower side of the side surface w) of the packing container 100 is held a closed state by attaching an adhesive tape such as a packing tape or the like.

Next, the surrounding member 200 is placed inside the side surface w of the packing container 100. The surrounding member 200 can be installed close to the side surface w to such an extent that the side of the syringe 500 and the retaining tool 400 to be housed can be surrounded.

After the surrounding member 200 is installed on the side in the internal space of the packing container 100, the syringe 500 and the retaining tool 400 as housed objects are disposed at substantially the center portion in the planar direction XY of the packing container 100. The syringe 500 and the retaining tool 400 can be disposed in a state of forming a gap with the surrounding member 200 in the first direction X or the second direction Y.

After the syringe 500 and the retaining tool 400 are disposed in the internal space (housing space) of the packing container 100, the cold storage agent 300 is housed between the surrounding member 200 and the syringe 500 and the retaining tool 400. By disposing the surrounding member 200 and the cold storage agent 300 between the syringe 500 and the retaining tool 400 which are contents, the syringe 500 can be transported in a state of being cooled to an extent that the syringe 500 does not become supercooled.

Next, the flaps f on the side opposite to the bottom surface of the packing container 100 (the upper side of the side surface w) is retained in a closed state with an adhesive tape or the like and transported toward the destination.

### (Use Method of Retaining Tool)

Next, a use method of the retaining tool 400 will be described. The retaining tool 400 is taken out from the internal space of the packing container 100, and the container is thawed from −40° ° C. to room temperature in a state where the syringe 500 is inserted into the insertion portion 410. As a result, the temperature of the viscous material filled in the syringe 500 can be relatively slowly changed to room temperature to bring the viscous material in a usable.

As described above, the retaining tool 400 according to the present embodiment includes the insertion portion 410 and the solid portion 420. The insertion portion 410 is installed in the housing space Sp of the packing container 100, and is configured to allow insertion of the syringe 500 in a state where the syringe 500 including the material storage container 510 that houses the viscous material is erected. The solid portion 420 is provided around the insertion portion 410 and forms the insertion portion 410.

The solid portion 420 is formed from the first end portion 424 on the side where the insertion portion 410 is located to the second end portion 425 on the opposite side of the first end portion 424 in the direction in which the viscous material is inserted into the insertion portion 410, and is configured to contain non-crosslinked foamed polyethylene.

With such a configuration, it is possible to prevent a rapid temperature rise of the viscous material when the viscous material housed in the syringe 500 is thawed. Furthermore, it is possible to suppress air bubbles from entering the interface between the syringe 500 and the viscous material and to mitigate impact during transportation.

Furthermore, the solid portion 420 is formed in a layer shape in a direction in which the syringe 500 is inserted into the insertion portion 410. Therefore, the retaining tool can be formed into a shape suitable for containers of any size, and the need for a mold can be eliminated, so that the manufacturability of the retaining tool 400 can be improved or made favorable.

Furthermore, the solid portion 420 is configured to provide a flat surface on the second end portion 425. With such a configuration, the viscous material can be transported in a state where the retaining tool 400 is stably placed on the placement surface during transportation.

Furthermore, in the use of the retaining tool 400, the syringe 500 is configured to be thawed from −40° C. to room temperature in a state where the syringe 500 is inserted into the insertion portion 410 of the retaining tool 400. With such a configuration, it is possible to prevent a rapid temperature rise of the viscous material housed in the syringe 500 and to prevent or suppress air bubbles from entering the interface between the syringe 500 and the resin.

Furthermore, the syringe transport tool set 1 includes the surrounding member 200 and the cold storage agent 300. The surrounding member 200 is installed in the housing space Sp of the packing container 100, and surrounds the syringe 500 including the material storage container 510 that houses the viscous material.

The cold storage agent 300 can be installed between the syringe 500 and the surrounding member 200 in the housing space Sp, and includes the content 330 that refrigerates the syringe 500 and the content storage container 310 that houses the content 330. The surrounding member 200 includes the buffer member that mitigates an external force applied to the syringe 500 when the syringe 500 is transported, and does not include the retaining tool 400 that retains the syringe 500. The surrounding member 200 has a thermal conductivity of 0.022 W/m·K or less, and the content 330 is configured to have a melting point of −30° ° C. or less.

When the dry ice is installed in the transport container and the viscous material is transported as described above, the viscous material is supercooled. On the other hand, when the vacuum heat insulating material is used for the transport container, the weight becomes relatively heavy, and the viscous material that can be transported at a time becomes relatively small. On the other hand, with a configuration as described above, it is possible to prevent or suppress supercooling of the viscous material by using a material other than the vacuum heat insulating material. Furthermore, by transporting the viscous material using the syringe transport tool set 1, it is possible to suppress temperature fluctuation of the viscous material with respect to temperature fluctuation of the outside air.

Furthermore, the content storage container 310 is configured such that the surfaces 311 to 316 have a flat surface. Therefore, the cold storage agent 300 can be installed in various postures with respect to the packing container 100, and the restriction caused by installing the cold storage agent 300 in the packing container 100 can be reduced to facilitate transportation of the syringe 500.

Furthermore, in the transportation method using the syringe transport tool set 1, the syringe 500 is disposed in the housing space Sp of the packing container 100, and the cold storage agent 300 is disposed in the housing space Sp so as to surround the syringe 500. Then, the surrounding member 200 is disposed in the housing space Sp so as to surround the syringe 500 via the cold storage agent 300.

With such a configuration, it is possible to prevent or suppress a change in product performance of the viscous material housed in the syringe 500 during transportation due to supercooling, and to prevent crystallization, separation, and sedimentation of the viscous material due to supercooling.

### (Experiment 1)

Next, the temperature change and the like at the time of thawing of the viscous material using the retaining tool was confirmed, and thus will be described.

In Experiment 1, the temperature change of the viscous material when the viscous material was thawed using two kinds of retaining tools was confirmed. The viscous material used in the experiment is an epoxy resin, and specifications of the syringe are as follows: a capacity of 70 cc, a total length of 223.7 mm, an outer diameter φ of 26.5 mm, and an external dimension of an insertion portion of 45 mm×30 mm, manufactured by Musashi Engineering, Inc.

The retaining tool according to the used comparative example is configured as illustrated in FIG. 5, and is a retaining tool in which dimensions of length, width, and height are 120 mm×220 mm×220 mm, 10 syringes can be set, and the inside is a hollow paper retaining tool. On the other hand, the retaining tool according to the example is a retaining tool in which dimensions of length, width, and height are 120 mm×220 mm×220 mm, 10 syringes can be set, the inside is solid, and non-crosslinked polyethylene (SUNTEC FOAM Q35) in which three layers are formed in the height direction is formed in an overlapping manner.

In Experiment 1, 10 syringes were set in the retaining tools according to the comparative example and the example, and the temperature change of the viscous material when the ambient temperature was changed from −40° ° C. to 20° C. corresponding to room temperature was confirmed. The temperature was measured using a data logger in a thermostatic bath set at 20° C. with a thermocouple attached to the center of the syringe in the height direction. Furthermore, in this experiment, the viscous material was thawed by the retaining tools according to the example and the comparative example, and it was confirmed whether or not the bead formed by the applied viscous material when applied from the dispenser was continuous. This is because, if air bubbles are generated at the time of thawing, it is considered that the bead cannot be continuously applied when being applied from the dispenser.

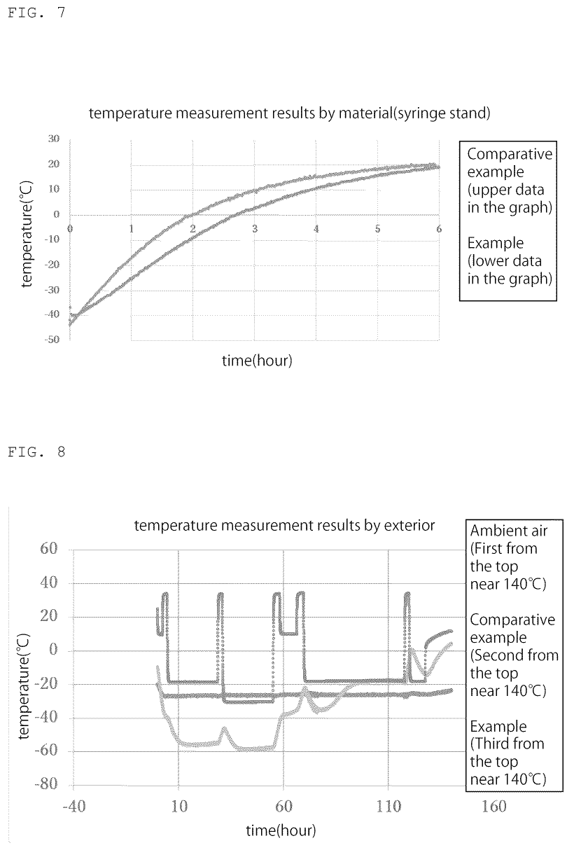

As the experimental results, as described above, the temperature changes of the comparative example and the example were confirmed, and whether air bubbles were generated in the viscous material such as the syringe or the like was visually confirmed in a state where the viscous material was housed in the syringe. Furthermore, application of the viscous material thawed using the retaining tools according to the example and the comparative example was performed using a dispenser in which the tip shape of the discharge portion was a needle (30 G), and whether or not the formed bead shape was discontinued was visually confirmed. The above test is also called a discharge test or the like, and air bubbles inside the viscous material that cannot be visually confirmed by appearance are confirmed by directly blowing air into the syringe to discharge the entire amount of the viscous material. Hereinafter, a graph of temperature changes when the syringes housing the viscous material in the retaining tools according to the comparative example and the example were thawed from −40° ° C. to room temperature is illustrated in FIG. 7. In the graph, located on the upper side corresponds to the comparative example, and located on the lower side corresponds to the example.

It could be confirmed that the temperature change from −40° C. to the room temperature was rapid in the paper retaining tool according to the comparative example as compared with the retaining tool according to the example. Furthermore, in the retaining tool according to the comparative example, air bubbles could be visually confirmed inside the syringe in the discharge test, whereas in the retaining tool according to the example, air bubbles could not be confirmed inside the syringe.

In addition, it could be confirmed that in the application of the viscous material from the dispenser, the application breakage of the bead of the viscous material occurred in the specification according to the comparative example, whereas in the specification according to the example, the application breakage of the viscous material did not occur, and the bead was continuous. From the above, it could be confirmed that the viscous material thawed according to the specifications of the example could be attached to a dispenser or the like and brought into a usable (dischargeable) state.

### (Experiment 2)

Hereinafter, an experiment related to transportation of a viscous material was performed, and thus will be described. FIG. 8 is a graph illustrating a temperature change of the viscous material with respect to a temporal change of an outside air temperature when the syringe housing the viscous material is transported using the transport tool sets according to an example and a comparative example in Experiment 2.

In Experiment 2, as a comparative example, a state was provided in which polystyrene foam, dry ice, and a viscous material housed in a syringe as a transportation target were installed in a cardboard packing container, and the packing container was installed in a thermostatic chamber in which the outside air temperature was set as described in FIG. 8 by a program for 140 hours. The melting point of the dry ice is −56.6° C. As polystyrene foam, one polystyrene foam having an outer size of 370 mm×496 mm×437 mm and a thermal conductivity of 0.04 W/m·K was used. As the dry ice, 14 pieces of dry ice having a weight of 500 g per one piece were used. As the syringe, a syringe made of polypropylene manufactured by Musashi Engineering, Inc. and having a capacity of 70 cc, a total length of 223.7 mm, an outer shape of q 26.5 mm, and a flange outer shape of 45×30 mm was used.

Furthermore, as an example, a state was provided in which polystyrene foam (corresponding to surrounding member, thermal conductivity is 0.022 W/m·K), a cold storage agent, and a viscous material housed in a syringe as a transportation target were installed in a cardboard packing container, and the packaging container was placed in a thermostatic bath for 140 hours. The melting point of the content of the cold storage agent is −30° C. The viscous material used is an epoxy resin.

The temperature of the viscous material housed in the syringe was measured in a state where the viscous material was installed in a thermostatic bath for 140 hours, and it was confirmed whether the measured temperature of the viscous material could be maintained within a range of −40° C. to −20° C., which did not affect or hardly affect the characteristics of the viscous material. Experimental results of the example and the comparative example are illustrated in FIG. 8.

In FIG. 8, the first line from the top at a temperature of 140° C. corresponds to the outside air (temperature), the second line from the top corresponds to the comparative example, and the third line from the top corresponds to the example. As can be seen from the graph, in the comparative example, it could be confirmed that the temperature was lower than −40° C. until 60 hours elapsed from the start, that is, so-called supercooling, and the temperature became higher than −20° ° C. after 120 hours elapsed.

On the other hand, it could be confirmed that the temperature could be maintained from −20° ° C. to −40° C. from the beginning to the end in the specification according to the example. That is, it could be confirmed that there was a high possibility that the product performance of the viscous material was affected during transportation in the specification according to the comparative example, while there was a high possibility that transportation could be performed without affecting the product performance of the viscous material in the specification according to the example. Note that, as the surrounding member according to the example, since supercooling can be prevented even in the vacuum heat insulating material, it is conceived that the surrounding member can be established even with thermal conductivity thereof being preferably 0.002 (more preferably 0.01) W/m·K, which is about the same as the thermal conductivity of the vacuum heat insulating material.

Note that the present application is based on JP 2021-069244 filed on Apr. 15, 2021, the disclosure of which is incorporated herein by reference in its entirety.

### REFERENCE SIGNS LIST

100 packing container
200 surrounding member (buffer member)
300 cold storage agent
310 content storage container
400 retaining tool
410 insertion portion
420 solid portion
421 first layer
422 second layer
423 third layer
424 first end portion
425 second end portion
500 syringe
510 material storage container
520 lid portion

1. A retaining tool comprising:
an insertion portion that is installed in a housing space of a packing container and allows insertion of a syringe including a material storage container that houses a viscous material in a state where the syringe is erected; and a solid portion that is provided around the insertion portion and forms the insertion portion,
wherein the solid portion is formed from a first end portion on a side where the insertion portion is located to a second end portion on an opposite side of the first end portion in an insertion direction in which the viscous material is inserted into the insertion portion, and contains non-crosslinked foamed polyethylene.

2. The retaining tool according to claim 1, wherein the solid portion is formed in a layer shape in the insertion direction.

3. The retaining tool according to claim 1, wherein the solid portion is provided with a flat surface at the second end portion.

4. A use method of a retaining tool comprising thawing the syringe according to claim 1 from −40° C. to room temperature in a state where the syringe is inserted into the insertion portion.

5. A syringe transport tool set comprising:
a surrounding member that surrounds the syringe according to claim 1; and
a cold storage agent that is installable between the syringe and the surrounding member in the housing space and includes a content that refrigerates the syringe and a content storage container that houses the content,
wherein the surrounding member includes a buffer member that mitigates an external force applied to the syringe when the syringe is transported and does not include the retaining tool according to claim 1,
the buffer member has a thermal conductivity of 0.022 W/m·K or less, and
the content is configured to have a melting point of −30 degrees or less.

6. The syringe transport tool set according to claim 5, wherein the content storage container has a flat surface.

7. A transportation method of a syringe, comprising:
disposing the syringe in the housing space of the packing container according to claim 5;
disposing the cold storage agent in the housing space so as to surround the syringe; and
disposing the buffer member in the housing space so as to surround the syringe via the cold storage agent.
